(19) United States
(12) Patent Application Publication
(10) Pub. No.: US 2024/0207156 A1
(43) Pub. Date: Jun. 27, 2024

(54) SULFATE-FREE DILUTABLE COMPACT PERSONAL CLEANSING COMPOSITION

(71) Applicant: The Procter & Gamble Company, Cincinnati, OH (US)

(72) Inventor: Geoffrey Marc WISE, Reading, OH (US)

(21) Appl. No.: 18/536,309

(22) Filed: Dec. 12, 2023

Related U.S. Application Data
(60) Provisional application No. 63/434,198, filed on Dec. 21, 2022.

(51) Int. Cl. A61K 8/46; A61Q 5/02; A61Q 5/12

(52) U.S. Cl. CPC A61K 8/466; A61Q 5/02; A61Q 5/12; A61K 2800/30; A61K 2800/5424; A61K 2800/5426; A61K 2800/5428; A61K 2800/596

(57) ABSTRACT

A stable, compact, low-viscosity personal care compositions that includes 20-40% total surfactant and is free of sulfated surfactants. The total surfactant includes at least 50% anionic surfactant and 10%-40% amphoteric surfactant. The anionic and amphoteric surfactants are present at ratio to provide the personal care composition with a Compact Viscosity of less than 2000 mPa-s and a Dilute Viscosity of greater than 2000 mPa-s. The personal care composition may optionally include an electrolytic viscosity modifier to tune the viscosity. The compact personal care composition is formulated to enable convenient dilution by a user and, when diluted, provide a desirable cleaning experience.

### FIELD OF THE INVENTION

The present disclosure relates generally to a low-viscosity, concentrated personal cleansing composition that transforms into a high-viscosity composition when diluted to a less concentrated composition. More specifically, the present disclosure relates to a low-viscosity, concentrated personal cleansing composition that includes a sulfate-free anionic surfactant and an amphoteric surfactant tailored to provide a suitably high viscosity when the concentrated composition is diluted with water by a user.

### BACKGROUND OF THE INVENTION

Personal cleansing compositions such as shampoos and body washes are common household products used by many consumers. Such compositions are commonly sold as a liquid or gel stored in a plastic bottle or tube, which is configured to conveniently dispense the composition. However, the negative environmental impact of plastic packaging continues to be a cause of concern for environmentally conscious consumers and companies. Thus, some manufacturers have turned to selling concentrated products (i.e., products that contain less water), to reduce the amount of plastic packaging used in their products.

While making and selling products in concentrated form may seem like a good way to reduce the amount of plastic packaging, at least some consumers do not like using concentrated products directly on the body, perceiving such products to be “too harsh”. They would prefer diluting the product at home (i.e., adding water to the concentrate and mixing), but concentrated products are often too thick to dilute homogeneously, which may result in poor product aesthetics (e.g., cloudy appearance), and concentrated product viscosities after dilution are too thin to be easily applied to the body. Accordingly, there remains a need for a concentrated personal cleansing composition that is convenient to dilute (e.g., pourable) and use and provides desirable in-use characteristics (e.g., homogeneous, foaming, cleaning, richness) to promote a reduction in plastic waste.

US20210220243 discloses a pourable concentrated surfactant composition with a lamellar phase that thickens and transforms to an isotropic phase upon dilution. According to US20210220243, the composition can be used as a concentrate in small volumes and diluted as used, or it can be diluted with water in a refill package to reduce plastic waste. These compositions use fatty acids, based on acyl isethionate, to drive the composition into a lamellar phase that maintains its viscosity upon dilution. However, the use of fatty acids has drawbacks. For example, fatty acids can form soap scum during rinsing, particularly in hard water, leading to objectionable consumer performance. Further, the shear-thinning nature of the lamellar phase can make it more difficult for a user to dilute the concentrated composition compared to a concentrated composition that is only modestly shear-thinning or exhibits near-Newtonian behavior. Indeed, most users want a thick, rich composition, not a thin, runny composition.

In another example, U.S. Pat. No. 11,129,783 discloses a stable, concentrated hair care composition with a low viscosity that can be delivered to hair in a variety of forms, including as a foam. However, not all consumers prefer foamed compositions, and when the concentrated compositions of U.S. Ser. No. 11/129,783 are diluted and applied as a conventional shampoo, the resulting compositions do not have the desired thickness.

Conventional personal care compositions such as shampoos and body washes commonly use sulfated surfactants to provide a cleaning benefit to skin and/or hair. However, there is a common perception amount some consumers that sulfated surfactants are harsh on skin and hair. Thus, it would be desirable to formulate dilutable concentrates that are substantially free of sulfated surfactants.

Sulfate-free composition present formulation challenges. For example, sulfate-free surfactant systems can be difficult to thicken without polymeric thickeners or agents that drive the formation of a lamellar phase. However, both of these approaches are unsuitable for formulating a sulfate-free dilutable concentrate. In particular, formulating with a suitable amount of polymeric thickener to provide a diluted compositions with the desired viscosity, will generally result in the concentrated composition being too thick to process. And agents that drive the formation of a lamellar phase will result in non-Newtonian (shear thinning) compositions that are much harder to dilute than a micellar or isotropic composition that is near-Newtonian.

Another drawback of sulfate-free surfactants is their higher cost relative to sulfated surfactants. Within the class of sulfate-free surfactants, alkyl olefin sulfonates may be preferred due to their high cleaning performance, wide availability, and low cost compared to other sulfate-free surfactants. However, the use of alkyl olefine sulfonates above 20% generally results in a compact composition with a viscosity that is too high to conveniently process (e.g., too thick for a user to conveniently pour). Additionally, it is generally accepted in the art that combinations of alkyl olefin sulfonate surfactants and certain amphoteric surfactants (e.g., cocamidopropyl betaine) do not build sufficient viscosity, for example, as disclosed in JP2021523172A.

Accordingly, it would be desirable to provide a sulfate-free, low-viscosity, compact personal cleansing composition that can be diluted by a user to provide a high-viscosity, dilute personal care composition. It would also be desirable to provide a low-viscosity, concentrated personal care composition that does not require extrinsic viscosity modifiers to provide the high-viscosity, dilute composition.

### SUMMARY OF THE INVENTION

An aqueous personal care composition, comprising at least 20% total surfactant, in which more than 50% is an anionic alkyl olefin sulfonate surfactant and 10% to 40% is an amphoteric surfactant. The personal care composition has a Compact Viscosity of less than 2000 mPa-s and a Dilute Viscosity of greater than 2000 mPa-s, according to the Viscosity method. The personal care composition is substantially free of sulfated surfactants.

### DETAILED DESCRIPTION OF THE INVENTION

Environmentally friendly products that use less plastic are in demand. Compact or concentrated personal cleansing compositions contain less water than their pre-diluted counterparts, and thus may need less plastic packaging. However, compact compositions present formulation challenges. For example, compact shampoo formulas can exhibit a relatively sharp boundary with regard to the amount of surfactant that can be added before the viscosity becomes too thick for processing. Compact personal care compositions need to be thin enough to be processed, but thick enough upon dilution to provide a desirable in-use cleaning experience. Moreover, the formulation problems need to be solved without increasing product cost and complexity. Conventional pre-diluted personal care compositions thin upon dilution with water, and so do not provide any guidance to help solve the aforementioned formulation problems.

Surprisingly, it has now been found that particular combinations of alkyl olefin sulfonate (“AOS”) with amphoteric surfactants and, optionally, an electrolyte, can help solve the aforementioned problems. It has also been found that higher concentrations of AOS can be used than previously thought feasible for compact compositions. As an additional unexpected benefit, the compositions herein do not require hydrotropes, fatty acids or water miscible solvents to provide the desired viscosity. This is beneficial because these types of materials can be expensive, provide objectionable performance (e.g., formation of soap scum) and they can reduce the ability of a composition to thicken upon dilution.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise and may be shown as “wt %”. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges not expressly stated, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Compact composition” means a composition that includes more than 20% surfactant.

“Compact viscosity” means the viscosity of a compact composition as described herein.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Dilute composition” means a compact composition that has been diluted by at least one part water per two parts of compact composition (i.e. to less than two thirds of the compact composition's surfactant concentration).

“Dilute viscosity” means the viscosity of a dilute composition as described herein.

“Personal cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material (e.g., less than 2%, 1% or even less than 0.5%), by weight of the composition or ingredient. “Free of” means a composition or ingredient comprises 0% of a subject material.

“Sulfate-free surfactants” means surfactants that are devoid of a sulfate moiety.

### Personal Care Composition

The personal care compositions herein are provided in the form of a compact composition that is intended to be diluted with water by a user prior to use. The compact personal care compositions have a low viscosity to enable a user to more easily pour the compact composition into a container for dilution. The compact compositions include a surfactant system, an aqueous carrier (e.g., water) and, optionally, an electrolytic viscosity modifier. The surfactant system exploits a specific combination of anionic and amphoteric surfactants to help provide the desired composition viscosity. The viscosity of the compact composition and dilute composition may be tuned by adding a specific amount of an electrolytic viscosity modifier. The personal care compositions can be made using conventional methods for making compositions of the type desired (e.g., shampoo or body wash).

The dilute personal care composition may be used to clean and/or condition hair or other body surfaces such as skin. The personal care composition may be provided in the form of liquid soap, body wash, shampoo or conditioner, for example. The personal care compositions herein may be formulated as cosmetic, cosmeceutical, or pharmaceutical (therapeutic) compositions, and may be leave-on compositions or rinse-off compositions.

In some aspects, the compact compositions may diluted with water at a ratio of composition to water of 3:1 to 1:4 (e.g., 2:1, 1:1, 1:2 or 1:3) to provide a 25% to 80% dilute composition. It may be desirable to formulate the compact composition such that the resulting dilute composition has a total surfactant level ranging from 12% to 15% (e.g., 13%). It is believed, without being limited by theory, that this range provides an excellent balance between pourability of the compact composition (i.e., Compact Viscosity) and cleaning performance of the dilute composition. Of course, it is to be appreciated that the amount of water used to dilute the compact is not particularly limited, and may be adjusted by a user, as desired, to provide a dilute composition with performance or aesthetic properties that suit the user.

At least some consumers prefer to use a personal care composition such as a shampoo or body wash that is clear. Thus, the compact personal care compositions herein may be formulated to become clearer upon dilution with water. In some aspects, the clarity of the dilute personal care composition may be selected to provide a signal to a user that the appropriate amount of water has been added to reach the intended dilution. For example, the compact composition may have a percent transmittance (% T) of less than 50% (e.g., less than 40%, 30%, 20%, 10% or even less than 5%). % T is a measure of the clarity of a composition and can be determined according to the Clarity method described in more detail below. When the compact composition is diluted with the appropriate amount of water (e.g., 25% to 80% dilution), the resulting dilute composition may have a % T of 80% or more (e.g., more than 85% or 90%, or even more than 95%), when measured according to the Clarity method.

In some aspects, a label or other indicia may be placed on the container intended to store the dilute composition, which can be, for example, a clear plastic or glass bottle. The label may be configured such that it is not clearly visible through the compact composition until the compact composition is diluted by a pre-determined amount (e.g., 3:1 to 1:4). Upon reaching the pre-determined dilution, the label or indicia can be clearly seen or read. Additionally or alternatively, a user may place an object (e.g., piece of paper, card, or picture) behind the package containing the dilute composition, and when the compact composition is diluted as intended, the object can be clearly seen through the container.

The personal care compositions herein may optionally include one or more additional ingredients commonly found in compositions of the type described, as long as the optional ingredient(s) do not interfere with the stability or efficacy of the composition. Some non-limiting examples of optional additional ingredients include preservatives, perfumes or fragrances, cationic polymers, coloring agents or dyes, conditioning agents, hair bleaching or coloring agents, moisturizers, foam boosters, emollients, humectants, pharmaceutical actives, vitamins or nutrients, sunscreens, sensates, plant extracts, nutrients, astringents, cosmetic particles, absorbent particles, adhesive particles, hair fixatives, fibers, skin lightening agents, skin tanning agents, antidandruff agents, exfoliating agents, enzymes, suspending agents, pH modifiers, anti-acne agents, sunscreens, hair growth or restorer agents, insect repellents, combinations thereof and the like.

The viscosity of the compact composition should be low enough to enable convenient pouring by a user for dilution, yet high enough to provide a desirable cleaning experience. “Low viscosity” means less than 2,000 mPa-s (e.g., less than 1,500 mPa-s, 1,000 mPa-s, 500 mPa-s or even less than 300 mPa-s), and “high viscosity” means greater than 2,000 mPa-s (e.g., greater than 2,500 mPa-s, 3,000 mPa-s, 5,000 mPa-s, 10,000 mPa-s, or even greater than 20,000 mPa-s), but typically less than 50,000 mPa-s. It is believed, without being limited by theory, that these viscosity ranges provide the composition with the desired pourability and cleaning experience.

The personal care compositions herein are stable after dilution, which is important because product instability is commonly perceived by consumers as a sign of poor product quality. Product instability may manifest as the formation of precipitate, phase separation, cloudy appearance and/or decrease in optical clarity of the composition. In some aspects, it may be important to select a suitable preservatives and/or counter-ion to enhance product stability. A method to determine Product stability is described in more detail below.

The personal care compositions may have a pH ranging from 2 to 10 (e.g., 3.0 to 7.0, 4 to 6.5, or even 4.5 to 6.0). It may be desirable to select a pH that helps the composition solubilize minerals and redox metals already deposited on the hair. Thus, the hair care composition can also be effective toward washing out the existing minerals and redox metals deposits, which can reduce cuticle distortion and thereby reduce cuticle chipping and damage. The pH can also be important for the effectiveness of the preservatives in the composition.

### Surfactants

The compact compositions herein include 20% to 40% (e.g., 25% to 40% or 30 to 35%) of a surfactant selected from anionic surfactants, amphoteric surfactants, zwitterionic surfactants, and combinations thereof. The type and amount of surfactant(s) present in the compact composition should be selected to provide the composition with good cleaning and foaming properties upon dilution and a Compact Viscosity of 2,000 mPa-s or less. The amount of surfactant disclosed herein refers to the amount of “active” and excludes the carrier (e.g., water) and excipients that are typically present in a commercially available surfactant product. Various examples and descriptions of detersive surfactants that may be suitable for use herein are disclosed in U.S. Pat. No. 8,440,605; US 2009/155383 and US 2009/0221463.

The personal care compositions herein include a sulfate-free anionic surfactants with an alkyl tail of 12 or more carbons (e.g., 13, 14, 15, 16, 17, 18, 19, 20, 21 or 22). That is, the surfactant molecule has an alkyl tail comprising a carbon chain of at least 12 carbon atoms. In some aspects, at least 50 wt % of the total surfactant in the compact composition (e.g., 50% to 100%, 55% to 95%, 60% to 90%, or even 65% to 80) is a sulfate-free anionic surfactant.

Sulfate-free anionic surfactants that may be suitable for use herein generally include branched or linear isethionates, sulfonates, ether sulfonates, sulfosuccinates, sulfoacetates, glycinates, sarcosinates, glutamates, alaninates, carboxylates, taurates, phosphate esters, salts of these (e.g., TEA, DEA, sodium, ammonium, and potassium salts) and combinations thereof. More specific examples of sulfate-free surfactants that may be suitable for use herein are disclosed in U.S. Publication No. 20190105246.

In some aspect, alkyl olefin sulfonates may be particularly suitable anionic surfactant for use herein. Alkyl olefin sulfonates are known compounds, for example, as described in Ullmann's Encyclopedia of Industrial Chemistry or in U.S. Pat. No. 8,211,850. Alkyl sulfonates have the general formula:

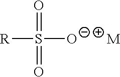

Where:
R=C8-C24 alkyl (linear or branched, saturated or unsaturated) or mixtures thereof, and
M⁺=monovalent cation.

In some aspects, the alkyl olefin sulfonate may be an α-olefin sulfonate, which typically includes a mixture of alkene sulfonates (formula I) and hydroxyalkyl sulfonates (formula II). Some non-limiting examples of alkyl olefin sulfonates that may be suitable for use herein include sodium C13-17 alkane sulfonate, sodium C12-14 olefin sulfonate and sodium C14-16 olefin sulfonate.

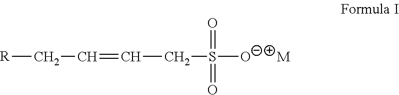

Where: R=C8-C18 alkyl, and M⁺=monovalent cation

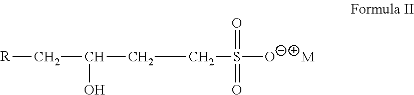

Where: R=C4-C18 alkyl and M⁺=monovalent cation.

### Amphoteric Surfactant

The surfactant may include 5% to 50% of an amphoteric surfactant (e.g., 10% to 40%, 15 to 35% or even 20 to 30%), based on the total weight of surfactant in the composition. Some non-limiting examples of amphoteric detersive surfactants that may be suitable for use herein include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Some specific examples of amphoteric detersive surfactants include cocoamphoacetate, cocoamphodiacetate, lauroamphoacetate, lauroamphodiacetate, cocamidopropyl betaine, cocamidopropyl hydroxysultaine alkanolamides (e.g., cocamide monoisopropanolamide) and mixtures thereof.

### Optional Additional Surfactants

The personal care composition may include optional additional surfactants to provide desired cleaning and lather performance. Any optional additional surfactants should be physically and chemically compatible with the other components of the personal care compositions described herein and should not otherwise unduly impair product stability, aesthetics, or performance. The amount of optional additional surfactants is not particularly limited and may be present in the compact composition at, for example, 5% to 25%, based on the total weight of surfactant (e.g., 8% to 20% or 9% to 15%).

### Viscosity Modifier

The personal care compositions herein may include 0.1% to 10% of a viscosity modifier (e.g., 0.5% to 8%, 1% to 5%, or 2% to 4%). Electrolytic viscosity modifiers may be particularly suitable for use herein because they are generally cheap, readily available and safe to use. The electrolytic viscosity modifier, when present, can be selected to work in conjunction with the amphoteric surfactant to “tune” the viscosity of the compact and dilute compositions. product. Surprisingly, for a particular combination of linear and branched anionic surfactants, there is a specific range of amphoteric surfactant and electrolyte levels that deliver the desired viscosity profile. For example, the concentration of electrolytic viscosity modifier in the dilute composition may be selected to deliver a surfactant microstructure (e.g., elongated micelles) with high viscosity at 13% surfactant. In this example, formulating to achieve the desired electrolyte level at 13% in the dilute composition implies a significantly higher electrolyte level in the concentrate. Thus, it can be important to balance the ingredients and ingredient levels in the compact composition in a way that provides the desired viscosity and cleansing properties in the dilute composition, which may not be intuitive.

Some non-limiting examples of electrolytic viscosity modifiers include sodium, ammonium and potassium salts with appropriate counter ions. In some aspects, sodium chloride and ammonium chloride may be particularly suitable.

In some aspects, the personal care compositions herein are substantially free of, or free of, hydrotropes, water miscible solvents, fatty acids, fatty alcohols and/or polymeric thickening agents commonly used to modify the viscosity of conventional compositions. This is desirable because these types of viscosity modifiers tend to be more expensive, exhibit consumer objectionable characteristics and/or do not provide a suitable Compact Viscosity or Dilute Viscosity.

Some non-limiting examples of water miscible solvents include dipropylene glycol, tripropylene glycol, diethylene glycol, ethylene glycol, propylene glycol, glycerin, 1,3-propane diol, 2,2-propanediol, 1,2-butanediol, 1,3-butanediol, 1,4-butanediol, 2,3-butanediol, and 2-methyl-2,4-pentanediol.

Some non-limiting examples of hydrotropes include alkali metal or ammonium salts of lower alkyl benzene sulphonates such as sodium xylene sulfonate (SXS), sodium cumene sulphonate, sodium toluene sulphonate and mixtures thereof.

Some non-limiting examples of fatty acid thickeners include lauric acid and stearic acid.

Some non-limiting examples of polymeric thickeners include acrylic thickening polymers such as sodium polyacrylate, polyethylacrylate, polyacrylamide, crosslinked (meth)acrylic acid homopolymers (e.g., products sold under the names Carbopol 980, 981, 954, 2984 and 5984 by Goodrich or products sold under the names Synthalen M and Synthalen K by the company 3 VSA) and crosslinked copolymers of (meth)acrylic acid and of a C1-C6 alkyl acrylate, and especially crosslinked copolymers of methacrylic acid and of ethyl acrylate or of acrylic acid and of ethyl acrylate (e.g., products sold under the names Viscoatex 538C by Coatex, Aculyn 33 by Rohm & Haas, or Carbopol Aqua SF-i by Noveon).

Additional non-limiting examples of viscosity modifiers can be found in US 20170157008 and U.S. Pat. No. 4,741,855.

### Conditioning Agent

The personal care composition herein may optionally include a conditioning agent. Conditioning agents include materials that are used to give a particular conditioning benefit to hair and/or skin. The conditioning agents useful in the hair care compositions of the present invention typically comprise a water-insoluble, water-dispersible, non-volatile, liquid that forms emulsified, liquid particles. The conditioning agent may present in the compact composition at 0.05% to 20% (e.g., 0.1% to 15%, 0.2% to 10%, 0.3% to 3%, or even 0.5% to 2%). Some non-limiting examples of conditioning agents that may be particularly suitable for use herein include silicones, aminosilicones, and cationic polymers such as cationic starch, cellulose and guar. Some non-limiting examples of conditioning agents are disclosed in U.S. Publication Nos. 20180078476 and 20190105247.

When the personal cleansing composition includes a cationic conditioning polymer, it may be desirable to minimize the amount of inorganic salt in the composition. Inorganic salt is sometimes added to sulfated surfactant-based cleansing formulations to thicken the product. However, high levels of inorganic salt in a sulfate-free formula in the presence of cationic conditioning polymer can cause product instability due to formation of a gel-like surfactant-polymer complex known as a coacervate. Maintaining low inorganic salt concentration (e.g., less than 1%) solves the instability issue in sulfate free formulations that include an anionic surfactant and high molecular weight cationic polymer.

### Anti-Dandruff Agents

The personal care composition herein may optionally include an anti-dandruff agent. As can be appreciated, the formation of a coacervate when the dilute composition is used as intended can facilitate deposition of an anti-dandruff agent to the scalp. Some non-limiting examples of anti-dandruff agents include azoles (e.g., climbazole, ketoconazole, itraconazole, econazole, and elubiol), hydroxy pyridines (e.g., piroctone olamine, ciclopirox, rilopirox, and MEA-hydroxyoctyloxypyridinone), kerolytic agents such as salicylic acid and other hydroxy acids, strobilurins such as azoxystrobin, metal chelators such as 1,10-phenanthroline and hinokitiol, pyridinethione particulates such as zinc pyrithone, piroctone olamine, sulfur and combinations of these.

### Opacifier

The personal care composition herein may include 0.01% to 5% of an opacifier or pearlescing agent. The opacifier or pearlescing agent is not particular limited, but should not undesirably affect the clarity (% T) of the dilute composition. Some non-limiting examples of opacifiers include polystyrene particles and dispersions with a particles size of 0.05 microns to about 0.45 micron (e.g., EUPERLAN® 810 or LYTRON® 621 and 631), styrene PVP copolymers, titanium dioxide, mica, long chain (C16-C22) acyl derivatives such as glycol or polyethylene glycol esters of fatty acid having 16 to 22 carbon atoms and up to 7 ethyleneoxy units (e.g., TEGO-PEARL®), alkanolamides of fatty acids, having from about 16 to about 22 carbon atoms, preferably about 16 to 18 carbon atoms such as stearic monoethanolamide, stearic diethanolamide, stearic monoisopropanolamide and stearic monoethanolamide, alkyl (C16-C22) dimethyl amine oxides such as stearyl dimethyl amine oxide and combinations thereof.

### Kit

The compact personal care compositions herein may be sold as part of a kit that includes a primary container that contains the compact composition, and a secondary container that is suitable for receiving and storing at least a portion of the compact composition for dilution. The configuration of the containers is not particularly limited. The containers may be the same or different sizes and made of the same or different materials, which may be disposable or reusable (e.g., clear plastic or glass). The first and second containers may be configured in a variety of ways to facilitate use of the product. For example, the first and/or second containers may include markings or indicia to show the proper levels of personal care composition for dilution and/or use.

Of course, it is to be appreciated that the compact composition can also be sold as a standalone product.

### Method of Use

The compact personal care compositions herein are intended to be diluted prior to use. In some aspects, to make a dilute composition for use, a portion (e.g., 10%, 20%, 25%, 33%, or 50%) of the compact composition contained in the primary container is poured into a secondary container of sufficient size to hold the entire dilute composition. Water is added to the second container to dilute the compact composition to the appropriate amount (e.g., 1:1, 2:1, 3:1, 4:1, 5:1, 6:1, 7:1, 8:1, 9:1 or even a 10:1 dilution). It may be desirable to dilute the compact composition such that the dilute composition comprises 13% total surfactant. Of course, it is to be appreciated that the compact composition may be diluted to a greater or lesser degree, as desired, for example to personalize the viscosity or cleaning power of the dilute composition. After the appropriate amount of water is added to the compact composition, the mixture is shaken by hand until the dilute composition appears homogeneous. In some aspects, the mixture is shaken for at least 5 seconds (e.g., 10, 20, 30 45, or even 60 or more seconds).

Once the compact composition is diluted, the diluted composition can be used in a conventional manner for cleansing and/or conditioning the hair and/or skin. Effective amounts of the dilute composition for use generally range from 1 g to 50 g (e.g., 1 g to 20 g). The method of cleaning hair or skin includes applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. In some aspects, the method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### Methods

### Viscosity Method.

This method can be used to determine the dynamic viscosity of the compact and dilute personal care compositions herein. The viscosity is measured using a Brookfield Rheometer R/S Plus™ brand cone and plate controlled-stress rheometer (or equivalent) according to the manufacturer's instructions. The cone (Spindle C-75-1) has a diameter of 75 mm and 1° angle. The viscosity is determined using a steady state flow experiment at a constant shear rate of 2 s⁻¹ at a temperature of 26.5° C. The sample size is 2.5 ml and the total measurement reading time is 3 minutes.

### Clarity Method (% T).

This method can be used to determine the clarity of the compact and dilute personal care compositions herein. The method uses a spectrophotometer (e.g., SpectraMax M-5 available from Molecular Devices or equivalent) to measure the percentage of light transmitted through the sample (% T). Higher % T values indicate clearer compositions. To determine % T, a sample is prepared in a suitable container (e.g., a 96 well, visible flat bottom plate such as Greiner part #655-001), ensuring that no air bubbles are within the sample. The flat bottom plate is placed within the spectrophotometer and % T is measured at 600 nm using suitable software (e.g., Software Pro v.5™ software available from Molecular Devices).

### EXAMPLES

### Example 1: Example Shampoo Formulations

Table 1 provides 13 examples of the inventive compact personal care compositions described herein and a comparative example (C1) of anon-inventive composition. C1 demonstrates a conventional formulation technique of using a relatively high level of a hydrotrope (i.e., 4% dipropylene glycol) to lower the viscosity of the compact composition. The compositions are made as 100-gram to 400-gram batches by combining the ingredients in a laboratory mixer at ambient temperature and mixing for several minutes. Dilutions are performed by pouring approximately 60 g of the concentration composition into an 8-ounce glass jar, pouring in the appropriate amount of water to achieve a 13% final surfactant level, screwing a lid on the jar and then wrist-shaking the closed jar for approximately five to ten seconds.

TABLE 1

| | | Ex. 1 | Ex. 2 | Ex. 3 | Ex. 4 | Ex. 5 | Ex. 6 | Ex. 7 |
|---|---|---|---|---|---|---|---|---|
| Chemistry Type | Ingredient | Wt % | | | | | | |
| Alkyl olefin sulfonate | Sodium C14-C16 | 22.8 | 17.8 | 20.8 | 22.7 | 23.3 | 16.6 | 18.7 |
| | olefin sulfonate ¹ | | | | | | | |
| Other sulfate-free anionic | Isethionate | | | | | | | |
| surfactant | | | | | | | | |
| Other sulfate-free anionic | Taurate | | | | | | | |
| surfactant | | | | | | | | |
| Amphoteric surfactant | Cocamidopropyl | 4.2 | 3.2 | 6.2 | 8.3 | | | |
| | betaine | | | | | | | |
| Amphoteric surfactant | Sodium | | | | | 10.3 | 7.4 | 8.3 |
| | lauroamphoacetate | | | | | | | |
| Conditioning polymer | Cationic guar | | | | | | | |
| Conditioning polymer | Cationic cellulose | | | | | | | |
| Conditioning oil | PDMS silicone | | | | | | | |
| Opacifier | Ethylene glycol | | | | | | | |
| | distearate | | | | | | | |
| Viscosity adjuster | Sodium chloride | 9.8 | 8.9 | 10.4 | 8.3 | 3.9 | 2.8 | 5.0 |
| Viscosity adjuster | Ammonium | | | | | | | |
| | chloride | | | | | | | |
| Viscosity adjuster | Lauric acid | | | | | | | |
| Total surfactant | | 27 | 21 | 27 | 31 | 34 | 24 | 27 |
| Wt ratio of anionic surfactant | | 0.85 | 0.85 | 0.77 | 0.73 | 0.69 | 0.69 | 0.69 |
| to total surfactant | | | | | | | | |
| Compact Viscosity | | 565 | 669 | 271 | 425 | 657 | 1073 | 555 |
| Dilute Viscosity (13% | | 3600 | 11145 | 9793 | 6450 | 8210 | 8210 | 9072 |
| surfactant) | | | | | | | | |

| | | Ex. 8 | Ex. 9 | Ex. 10 | Ex. 11 | Ex. 12 | Ex. C1 |
|---|---|---|---|---|---|---|---|
| Chemistry Type | Ingredient | Wt % | | | | | |
| Alkyl Olefin Sulfonate | Sodium C14-C16 | 27.7 | 20.8 | 20.8 | 20.8 | 20.8 | |
| | olefin sulfonate ¹ | | | | | | |
| Other sulfate-free anionic | Isethionate | | | | | | 9.8 |
| surfactant | | | | | | | |
| Other sulfate-free anionic | Taurate | | | | | | 2.0 |
| surfactant | | | | | | | |
| Amphoteric | Cocamidopropyl | | | 4.2 | 3.2 | 6.2 | 21.2 |
| | betaine | | | | | | |
| Amphoteric | Sodium | 2.3 | 9.2 | | | | |
| | lauroamphoacetate | | | | | | |
| Conditioning polymer | Cationic guar | | | 0.6 | | | |
| Conditioning polymer | Cationic cellulose | | | | 0.6 | | |
| Conditioning oil | PDMS silicone | | | | | 1.2 | |
| Opacifier | Ethylene glycol | | | | | 1.5 | |
| | distearate | | | | | | |
| Viscosity adjuster | Sodium chloride | 14.3 | | 10.4 | 10.4 | 10.4 | 2.0 |
| Viscosity adjuster | Ammonium | | 2.3 | | | | |
| | chloride | | | | | | |
| Viscosity adjuster | Lauric acid | | | | | | 4.6 |
| Total surfactant | | 30 | 30 | 27 | 27 | 27 | 33 |
| Wt. ratio of anionic | | 0.92 | 0.69 | 0.77 | 0.77 | 0.77 | 0.36 |
| surfactant to total | | | | | | | |
| surfactant | | | | | | | |
| Compact Viscosity | | 1102 | 725 | 697 | 694 | 635 | 1504 |
| Dilute Viscosity (13% | | 9070 | 6520 | 7540 | 6350 | 8510 | 8481 |
| surfactant) | | | | | | | |

¹ Bio-Terge ® AS-40 available from Stepan Company

### Example 2: Comparative Non-Inventive Compositions

Table 2 shows examples of comparative compositions from U.S. Pat. No. 11,129,783 that do not exhibit suitable viscosity. The examples selected from US1129783 were recreated and diluted to 13% total surfactant. Lx. C13 was modified to remove the dipropylene glycol, a water miscible thickener, and Ex. C20 was modified to remove the sodium xylene sulfonate, a hydrotrope thickener. Reported values are the values listed in the patent. Measured values were determined according to the Viscosity Method. As can be seen in Table 2, these modifications did not result in a suitable dilute composition.

TABLE 2

| | | | | | | C13- |
|---|---|---|---|---|---|---|
| Chemistry Type | Ingredient | C1 | C4 | C7 | C13 | mod |
| Total surfactant | | 30 | 30 | 30 | 20 | 20 |
| Wt. ratio of linear | | 0.69 | 0.64 | 0.60 | 0.31 | 0.31 |
| anionic surfactant to | | | | | | |
| total anionic surfactant | | | | | | |
| Wt. ratio of amphoteric | | 0.13 | 0.27 | 0.33 | 0.13 | 0.13 |
| surfactant to total | | | | | | |
| surfactant | | | | | | |
| Linear anionic surfactant | SLE1S | 18 | 14 | 12 | 5.33 | 5.33 |
| Branched anionic | ST2S | 8 | 8 | 8 | 12 | 12 |
| surfactant | | | | | | |
| Amphoteric surfactant | CAPB | 4 | 8 | 10 | 2.67 | 2.67 |
| Amphoteric surfactant | Sodium lauro- | | | | | |
| | amphoacetate | | | | | |
| Conditioning polymer | Cationic guar | | | | | |
| Conditioning polymer | PEG 23M | | | | | |
| Viscosity modifier | Dipropylene | 4 | 4 | 4 | 2.67 | 0 |
| (water miscible solvent) | glycol | | | | | |
| Viscosity modifier | Sodium xylene | | | | | |
| (hydrotrope) | sulfonate | | | | | |
| Viscosity modifier | Glycerin | | | | | |
| (water miscible solvent) | | | | | | |
| | Reported | 2324 | 1110 | 751 | 278 | n/a |
| | Viscosity | | | | | |
| | Measured | 2566 | 1572 | 811 | 310 | 1308 |
| | Viscosity | | | | | |
| | (13% surfactant) | | | | | |
| | Dilute Viscosity | <10 | 237 | 1283 | <10 | <10 |
| | (13% surfactant) | | | | | |
| | | | | | | C20- |
| Chemistry type | | C15 | C16 | C18 | C20 | mod |
| Total surfactant | | 30 | 30 | 30 | 24 | 24 |
| Wt. ratio of linear anionic | | 0.67 | 0.31 | 0.69 | 0.75 | 0.75 |
| surfactant to total anionic | | | | | | |
| surfactant | | | | | | |
| Wt. ratio of amphoteric | | 0.20 | 0.13 | 0.13 | 0 | 0 |
| surfactant to total | | | | | | |
| surfactant | | | | | | |
| Linear anionic | SLE1S | 16 | 8 | 18 | 18 | 18 |
| Branched anionic | ST2S | 8 | 18 | 8 | 6 | 6 |
| Amphoteric | CAPB | 6 | 4 | 2 | | |
| Amphoteric | Sodium lauro- | | | 2 | | |
| | amphoacetate | | | | | |
| Conditioning polymer | Cationic guar | | | 0.4 | | |
| Conditioning polymer | PEG 23M | | | | 0.1 | 0.1 |
| Hydrotrope | Dipropylene | | | 4 | | |
| | glycol | | | | | |
| Hydrotrope | Sodium xylene | | 4 | | 2.5 | 0 |
| | sulfonate | | | | | |
| Hydrotrope | Glycerin | 4 | | | | |
| | Reported | 2170 | 982 | 2029 | 1960 | n/a |
| | Viscosity | | | | | |
| | Measure | 2812 | 985 | 2834 | 1070 | 676 |
| | Viscosity | | | | | |
| | Dilute Viscosity | <10 | <10 | <10 | <10 | <10 |
| | (13% surfactant) | | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compact personal care composition, comprising:
a) at least 20 wt % of total surfactant, by weight of the composition, comprising:
i) more than about 50 wt % of an anionic alkyl olefin sulfonate surfactant, by weight of the surfactant, and
ii) about 10 wt % to about 40 wt % of an amphoteric surfactant, by weight of the surfactant; and
b) water,
wherein the composition has a Compact Viscosity of less than about 2000 mPa-s and a Dilute Viscosity of greater than about 2000 mPa-s, according to the Viscosity Method, and the composition is substantially free of sulfated surfactants.

2. The composition of claim 1, wherein the anionic alkyl olefin sulfonate surfactant is an α-olefin sulfonate surfactant selected from sodium C12-14 olefin sulfonate, sodium C14-16 olefin sulfonate or a combination thereof.

3. The composition of claim 1, wherein the composition comprises 20% or more alkyl olefin sulfonate surfactant, based on the weight of the composition.

4. The composition of claim 1, wherein the amphoteric surfactant is selected from the group consisting of betaines, lauroamphoacetates, alkanolamides, and combinations thereof.

5. The composition of claim 1, further comprising a conditioning agent.

6. The composition of claim 5, wherein the conditioning agent is a cationic conditioning polymer.

7. The composition of claim 6, wherein the cationic conditioning polymer has a weight average molecular weight of about 50 kDa to about 2 MDa and a charge density of about 1 meq/g to about 2.5 meq/g.

8. The composition of claim 1, further comprising an electrolytic viscosity modifier.

9. The composition of claim 8, wherein the electrolytic viscosity modifier is sodium chloride, ammonium chloride or a combination thereof.

10. The composition of claim 1, further comprising an anti-dandruff agent selected from azoles, hydroxyl pyridones, zinc pyrithone, piroctone olamine, sulfur, ciclopirox, rilopirox, MEA-hydroxyoctyloxypyridinone, salicylic acid, azoxystrobin, 1,10-phenanthroline, or a combination thereof.

11. The composition of claim 1, wherein the composition is free of viscosity modifiers selected from the group consisting of hydrotropes, water miscible solvents, fatty acids and acrylic polymers.

12. The composition of claim 1, wherein the compact composition has a % T of less than about 50 according to the Clarity Method.

13. A method of making a dilute personal cleansing composition, comprising:
a) adding an amount of the compact personal cleaning composition of claim 1 into a container; and
b) adding water to the container to form a dilute personal care composition, wherein the amount of water added provides a ratio of composition to water of 3:1 to 1:4.

14. The method of claim 13, wherein the dilute composition has a % T of greater than about 75 according to the Clarity method.

15. The method of claim 13, wherein the dilute personal care composition has a viscosity that is at least 10% greater than a viscosity of the compact personal care composition, according to the Viscosity method.

16. The method of any one of claim 13, wherein the dilute personal care composition comprises elongated micelles.

17. The method of one of claim 13, wherein the dilute personal care composition has a % T of greater than about 75%, according to the Clarity method.

18. The method of one of claim 13, wherein the dilute composition comprises about 11% to about 15% total surfactant, preferably 12% to 14%, and more preferable 13%.

19. A method of cleaning hair, comprising:
a) dispensing the dilute personal cleansing composition of claim 13 onto a hand or implement;
b) applying the dilute personal care composition to a body surface; and
c) rinsing the dilute personal care composition from the body surface.

20. The method of claim 19, wherein the dilute personal care composition is applied to the body surface more than 1 minute after making the dilute composition.
